(19) United States
(12) Patent Application Publication
(10) Pub. No.: US 2011/0065161 A1
(43) Pub. Date: Mar. 17, 2011

(54) BIPOLAR SOLID STATE MARX GENERATOR

(75) Inventors: Alexis Kwasinski, Austin, TX (US); Sung Woo Bae, Austin, TX (US); Mark M. Flynn, Austin, TX (US); Robert E. Hebner, Austin, TX (US); Michael D. Werst, Manor, TX (US); Siddharth B. Pratap, Austin, TX (US); Aaron S. Williams, Round Rock, TX (US)

(73) Assignee: BOARD OF REGENTS, THE UNIVERSITY OF TEXAS SYSTEM, Austin, TX (US)

(21) Appl. No.: 12/881,748

(22) Filed: Sep. 14, 2010

Related U.S. Application Data
(60) Provisional application No. 61/242,371, filed on Sep. 14, 2009.

(51) Int. Cl. G05F 3/08; H02J 1/00; C12N 13/00; C02F 1/48; A23L 3/32

(52) U.S. Cl. 435/173.1; 323/311; 307/43; 210/748.01; 426/237

(57) ABSTRACT

A high-voltage bipolar rectangular pulse generator using a high efficiency solid-state boosting front-end and an H-bridge output stage is described. The topology of the circuit generates rectangular pulses with fast rise time and allows easy step-up input voltage. In addition, the circuit is able to adjust positive or negative pulse width, dead-time between two pulses, and operating frequency. The intended application for such circuit is algae cell membrane rupture for oil extraction, although additional applications include biotechnology and plasma sciences medicine, and food industry.

### TECHNICAL FIELD OF THE INVENTION

The present invention relates in general to the field of generating high voltage pulses, and more particularly to a bipolar solid state Marx generator that can produce both bipolar and unipolar high-powered rectangular pulses to distend and stress biological cells or non-thermally pasteurize/sterilize food and water.

### CROSS-REFERENCE TO RELATED APPLICATIONS

This patent application is a non-provisional application of U.S. patent application 61/242,371 filed on Sep. 14, 2009 entitled “Bipolar Solid-State Marx Generator”, which is hereby incorporated by reference in its entirety.

### STATEMENT OF FEDERALLY FUNDED RESEARCH

None.

### BACKGROUND OF THE INVENTION

Without limiting the scope of the invention, its background is described in connection with methods and devices for generating high voltage pulses and application of such devices in biological systems.

Pulse electric fields (PEF) are used, for example, to induce stress and mortality in biological cells and perform non-thermal food pasteurization/sterilization. Although there exists some controversy with respect to the correct term that should be used to describe the effects resulting from applying electric fields with different characteristics to cells [1], it can surely be said that these effects can be reversible or irreversible [2]. Reversible application of electric fields is used in many fields, including science, medicine, and biotechnology, in order to introduce proteins or molecules in cells [2]-[6] or to fuse two cells together [2]-[4]. Irreversible electric field application leads to cell rupture—a desired outcome in many applications, including food industry [7], public health [8], and water purification [9].

In many of these applications of high-power pulse generators, particularly in those involving irreversible processes, bipolar pulse generation has specially attracted attention because of its better process output over unipolar pulses [8], [10]. It is also desirable to achieve different output field intensities so the generator could be used both for reversible processes requiring lower field intensities and irreversible processes needing higher field intensities [11].

Cost effectiveness and high efficiency are difficult goals to achieve for high-voltage and high-power applications because of the severe requirements in terms of voltages and power usually demanded to pulse generators components. These requirements are one of the main disadvantages that prevent using the well known original design for high-power pulse generators patented by Erwin Otto Marx in 1923 [12] because of the many resistances in the discharge path. The same efficiency issues are observed in some recently proposed topologies [13].

An alternative in order to achieve higher efficiency is to use semiconductor-based circuit topologies, for which [14] presents the general design principles. Among those, one alternative is to have a cascade arrangement of inductors and capacitors [9] [15]; however, this design requires a high-voltage source and is not flexible enough for a broad set of applications. Some other previous works generate high voltage spikes by attempting to interrupt a flux in a magnetic core [16] [17], but these circuits have a complicated magnetic design, usually create significant stress on the switches, and, generally, do not produce square pulses. Several of the topologies previously suggested using semiconductor devices tend to have a large number of switches [18]-[25]. Since each of them tend to be costly, particularly because the intended applications require high-voltage and high-power, the entire design tends to be costly.

Other alternatives require having multiple sources [26] [27]. Since, only one source is usually available circuits with multiple sources tend to be impractical. However, one of these circuits [27] has an interesting arrangement based on an H-bridge configuration that allows a flexible output configuration without changing the circuit connections or components. On the contrary, the topology suggested in [28] requires a change off-line in the ground and load position in order to achieve pulses with different polarity, so only unipolar pulses can be generated. However, the input stage is composed of a cascade of boost cells in which the capacitors are charged at the input voltage level, yielding more reliable and less costly devices. Finally, U.S. Pat. No. 6,214,297 issued to Zhang and Qiu (2001) describes various designs in which a power source charges an energy storage component which discharges into a pulse transformer to a PEF treatment chamber or a series connected H-bridge configuration.

As a result, it follows that a design does not exist that simultaneously meets all the required conditions of flexibility, easily adjusted output, and efficiency required.

### SUMMARY OF THE INVENTION

The present invention provides a bipolar solid state Marx generator that is flexible, efficient and has an easily adjusted output that can, produce both bipolar and unipolar high-powered rectangular pulses to distend and stress biological cells or non-thermally pasteurize/sterilize food and water. For example, the present invention generates bipolar and rectangular pulsed waveforms suitable to extract oil by rupturing algae cells. Algal oils have an ultimate goal of providing an alternative source of transportation fuels, as fossil fuels costs increase due to diminishing reserves of easily extracted oil.

In one embodiment of the present invention, a bipolar high-power pulse generator includes a DC power source, a DC-DC converter connected to the DC power source, a H-bridge switching circuit connected in parallel with the DC-DC converter. The H-bridge switching circuit includes four switches (A+, A−, B+, B−) connected in a H configuration with a load connected across the bridge. A controller is connected to the DC-DC converter and the H-bridge switches (A+, A−, B+, B−). In one alternative embodiment, a diode (D_{A+}, D_{A-}, D_{B+}, D_{B-}) is connected in parallel with each switch (A+, A−, B+, B−) in the H-bridge switching circuit. In another alternative embodiment, the DC-DC converter includes two or more boost cells connected together, wherein each boost cell comprises a positive input node, a negative input node, a switch (Sᵢ) connected in series with an inductor wherein the series connected switch (Sᵢ) and inductor are connected in parallel with the positive and negative nodes, a diode connected in series with a capacitor wherein the series connected diode and capacitor are connected in parallel with the switch (Sᵢ) and the capacitor is connected in parallel with a positive output node and a negative output node. The generator delivers a positive pulse to the load whenever the switch (Sᵢ) is on, the H-bridge switches (A+, B−) are on, and the H-bridge switches (A−, B+) are off. The generator delivers a negative pulse to the load whenever the switch (Sᵢ) is on, the H-bridge switches (A−, B+) are on, and the H-bridge switches (A+, B−) are off.

The present invention also provides a method of treating one or more biological cells or a pumpable food within a treatment chamber by providing a bipolar high-power pulse generator. The bipolar high-power pulse generation includes (a) a DC power source, (b) a DC-DC converter connected to the DC power source, wherein the DC-DC converter comprises two or more boost cells connected together, wherein each boost cell comprises a positive input node, a negative input node, a switch (Sᵢ) connected in series with an inductor wherein the series connected switch (Sᵢ) and inductor are connected in parallel with the positive and negative nodes, a diode connected in series with a capacitor wherein the series connected diode and capacitor are connected in parallel with the switch (Sᵢ) and the capacitor is connected in parallel with a positive output node and a negative output node, (c) a H-bridge switching circuit connected in parallel with the DC-DC converter, wherein the H-bridge switching circuit comprises four switches (A+, A−, B+, B−) connected in a H configuration with the treatment chamber connected across the bridge, and (d) a controller connected to the DC-DC converter and the H-bridge switches (A+, A−, B+, B−). One or more pulses are delivered to the treatment chamber. A positive pulse is delivered whenever the controller sequentially turns the H-bridge switches (A+, B−) on, turns the switch (Sᵢ) on, turns the switch (Sᵢ) off, and turns the H-bridge switches (A+, B−) off. A negative pulse is delivered whenever the controller sequentially turns the H-bridge switches (A−, B+) on, turns the switch (Sᵢ) on, turns the switch (Sᵢ) off, and turns the H-bridge switches (A−, B+) off.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic illustration of the concept of a bipolar high power pulse generator in accordance with the present invention;

FIGS. 2A-2D show a switching strategy and simplified output voltage across the load for the bipolar high power pulse generator shown in FIG. 1;

FIGS. 3A-3C show three basic approaches for realization of a high-voltage source in the bipolar Marx generator of the present invention: a pulse forming network (FIG. 3A), a DC-DC converter and H-bridge series connection (FIG. 3B), and a DC-DC converter and H-bridge parallel connection (FIG. 3C);

FIG. 4 is circuit diagram illustrating a topology of the bipolar semiconductor Marx generator in accordance with one embodiment of the present invention;

FIGS. 5A-5D are operational modes timing diagrams for the bipolar solid-state Marx generator in accordance with one embodiment of the present invention;

FIGS. 6A-6E are circuit diagrams illustrating the operational stages of the bipolar solid-state Marx generator in accordance with one embodiment of the present invention;

FIG. 7 is a flow chart illustrating a method of treating one or more biological cells, water, or a pumpable food within a treatment chamber in accordance with one embodiment of the present invention;

FIG. 8 is a circuit diagram of a prototype bipolar solid state Marx generator in accordance with one embodiment of the present invention;

FIGS. 9A-9C are graphs showing simulated output voltages and inductor currents for the bipolar solid state Marx generator shown in FIG. 7;

FIGS. 10A-10D show simulated output voltages and switching strategy for the bipolar solid state Marx generator shown in FIG. 7;

FIGS. 11A-11D shows simulated output voltages for pulse patterns for the bipolar solid state Marx generator shown in FIG. 7;

FIGS. 12A-12B show a set-up for using the bipolar solid-state Marx generator of the present invention (FIG. 12A) and a close up of the circuit details (FIG. 12B);

FIG. 13 is an oscilloscope trace showing an undesirable voltage spike with opposite voltage at the beginning of the negative pulse;

FIG. 14 is an oscilloscope trace showing ringing and overvoltages caused by excessive fast switching for Sᵢ;

FIG. 15 is an expanded view of the area circled in FIG. 14;

FIG. 16 are equivalent traces to those in FIG. 14 but are obtained with an adequately fast switching for Sᵢ;

FIG. 17 shows switching signals, output current and voltage traces with an adequate choice time duration in each mode;

FIGS. 18A-C are debris microscopic image showing: an unpulsed control sample (FIG. 18A), a sample after 25 bipolar pulses (FIG. 18B), a sample after 50 unipolar pulses (FIG. 18C); and

FIG. 19 is a comparison chart of the unpulsed control sample, the sample after the bipolar pulses, and the sample after unipolar pulses.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides a bipolar solid state Marx generator that is flexible, efficient and has an easily adjusted output that can, produce both bipolar and unipolar high-powered rectangular pulses to distend and stress biological cells or non-thermally pasteurize/sterilize food. For example, the present invention generates bipolar and rectangular pulsed waveforms suitable to extract oil by rupturing algae cells. Algal oils have an ultimate goal of providing an alternative source of transportation fuels, as fossil fuels costs increase due to diminishing reserves of easily extracted oil.

Moreover, the present invention has many desired characteristics. For example, the present invention provides a flexible output configuration without modifying circuit components or layout in order to produce both single and a train of unipolar or bipolar pulses with adjustable pulse-width. In addition, different output field intensities can be achieved so the generator could be used both for reversible processes requiring lower field intensities and irreversible processes needing higher field intensities. The present invention also provides a cost effective and high efficient circuit design, which is an important requirement in the production of low-cost fuels. The circuit design also presents additional desirable features, such as fast rise time and easy step-up input voltage.

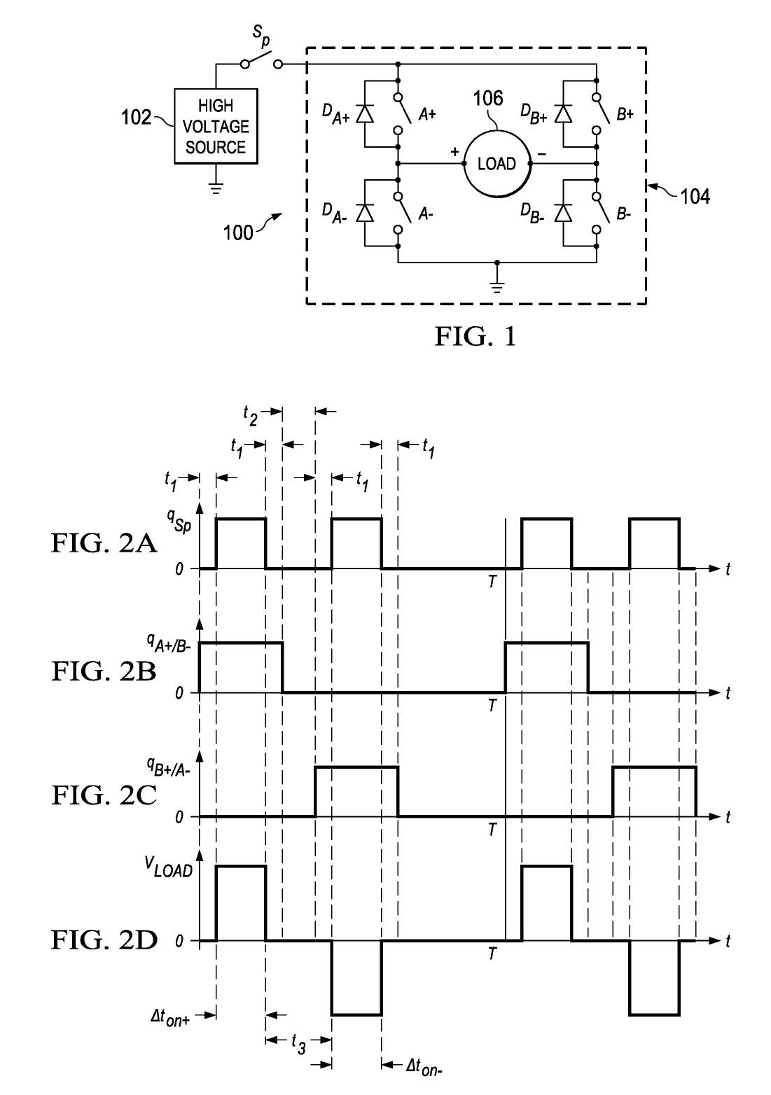

The concepts and operational principles of the bipolar high-power pulse generator in accordance with the present invention will now be described. FIG. 1 depicts the general concept for the bipolar high power pulse generator 100 of the present invention. The bipolar high power pulse generator 100 includes a high voltage source 102 connected in series with an input switch Sₚ and a H-bridge switching circuit 104. The H-bridge switching circuit includes four switches (A+, A−, B+, B−) connected in a H configuration with a load 106 connected across the bridge. The H-bridge switching circuit 104 allows a flexible configuration in order to achieve both bipolar and unipolar pulses with either polarity. The input switch Sₚ and the H-bridge switches (A+, A−, B+, B−) are operated by a controller (not shown), which can be a signal generator, a computer or other suitable control mechanism. Even with resistive loads, as is the dominant characteristic of algae cells, it is desirable to include freewheeling diodes (D_{A+}, D_{A-}, D_{B+}, D_{B-}) in parallel with the H-bridge switches (A+, A−, B+, B−) in order to provide a bidirectional current path for stray inductances or other undesirable events. 12. The load 106 may include a pulse electric field (PEF) treatment chamber containing one or more biological cells, water, or a pumpable food. Note that the PEF treatment chamber can be part of a constant flow treatment process. Moreover, the one or more biological cells may include bacterial cells, viral cells, algal cells, protozoal cells, plant cells, mammalian cells, animal cells or any combinations thereof. For example and as described below, algal cells release oil on lysis as a result of the pulses generated by the generator 100.

FIGS. 2A-2D detail the switching strategy required to achieve bipolar pulses. More specifically, FIG. 2A shows the timing for the input switch Sₚ, FIG. 2BA shows the timing for the H-bridge switches pair A+/B−, FIG. 2C shows the timing for the H-bridge switches pair A−/B+, and FIG. 2D shows the voltage V_{LOAD} across the load 106. This strategy is to alternate a load current path by dividing the conducting time of the high voltage source switch Sₚ indicated in FIG. 1 between the H-bridge switches pairs A+/B− and A−/B+. Hence, voltage polarity is controlled with the H-bridge switches, whereas current conduction is controlled with the input switch Sₚ. In order to achieve bipolar pulses, A+ and B− switches are turned on first, and then B+ and A− switches are turned on usually for an equal interval after A+ and B− are turned off. As FIG. 2A indicates, the switch Sₚ in FIG. 1 turns on only when one of the two opposing H-bridge switch pairs are commanded into the conducting state. As FIGS. 2B-2D also indicates, unipolar pulses operation is just a special case of bipolar pulse generation in which only one of the two H-bridge pairs of opposing switches are turned one. Thus, unless specified otherwise the analysis will focus on the bipolar case. Since it can be expected that in practice the commutation times for Sₚ and the H-bridge switches are different—Sₚ is assumed to be faster than the H-bridge switches—a delay time t₁ is deliberately added as indicated in FIG. 2A in order to avoid at the beginning of a pulse output voltage spikes of opposite polarity than the one for the desired pulse. In addition, a dead time t₂ needs to be added as indicated in FIG. 2B, in order to avoid shoot-through currents due to simultaneous conducting intervals of H-bridge switches in the same leg. Thus as indicated in FIG. 2D, the proposed bipolar pulse generator 100 has a total dead time t₃, resulting from adding the delay times t₂ and twice t₁.

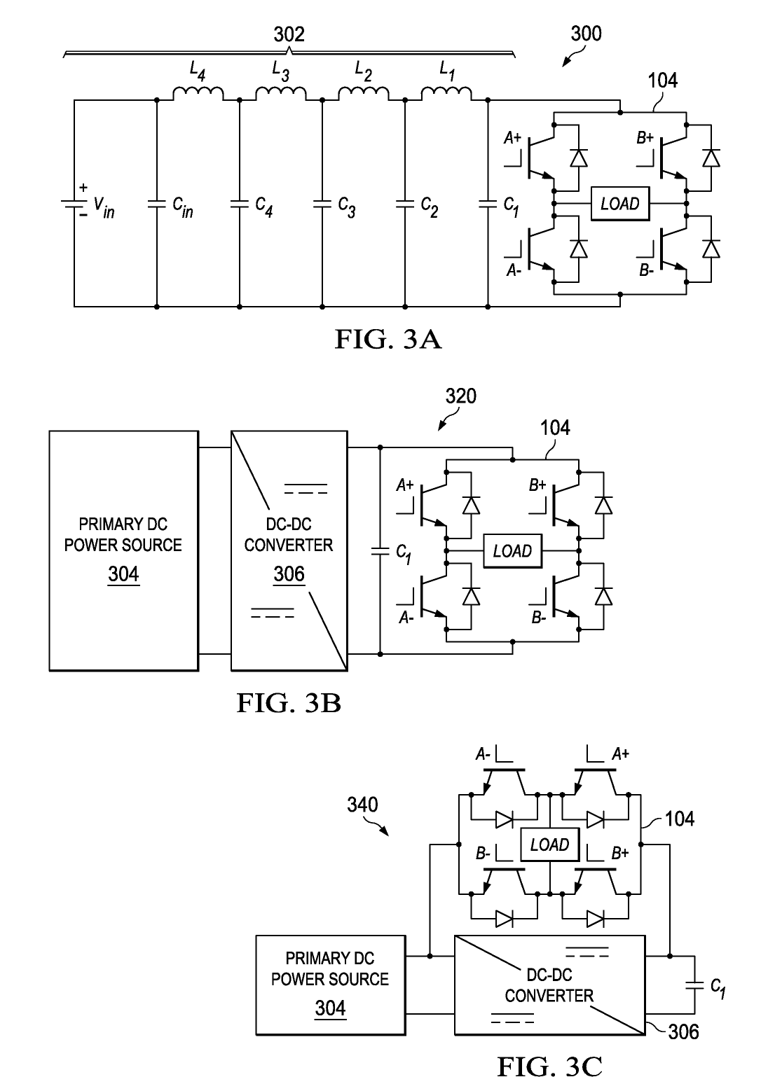

The proposed concept for a bipolar pulse generator 100 is completed with the analysis of the high-voltage source 102 indicated in FIG. 1. This high-voltage power source 102 acts as an energy buffer, storing it from a low-power source and, then, delivering it to the load 106 with a high-power short impulse. As previously discussed, there are several previously proposed options to build the high-voltage source stage, although most of them are inadequate for the application discussed in the present invention. Some general approaches to design this stage are represented in FIG. 3. Depending on the high voltage source 102 in FIG. 1, the proposed bipolar pulse generator 100 may be categorized into three topologies 300, 320, and 340 as indicated in FIGS. 3A-3C. More specifically, FIG. 3A shows a bipolar pulse generator 300 that uses a pulse forming network (PFN) 302 having a LC ladder architecture to produce rectangular impulses [15]. The H-bridge switching circuit 104 is connected in series with the PFN 302. However, this topology is not flexible to operate and requires a high-voltage input source itself because it is not able to step up the input voltage. Another approach is the bipolar pulse generator 320 in FIG. 3B with the output stage connected in parallel to the output capacitor C₁, i.e. in a series connection with a DC-DC converter 306 that step-ups the input voltage from a primary DC power source 304. The H-bridge switching circuit 104 is connected in series with the combined DC-DC converter 306 and output capacitor C₁ circuit. This approach usually requires an intermediate high-frequency transformer in the DC-DC converter 306 in order to achieve the desired step-up voltage conversion ratio [16]. However, this transformer tends to limit the pulses rising and dropping times due to its leakage inductances. Another approach is to utilize a Marx generator whose capacitors are charged in parallel and discharged in series to achieve high output voltage. As shown in FIG. 3C, the bipolar pulse generator 340 also includes a DC-DC converter 306 that step-ups the input voltage from a primary DC power source 304. The H-bridge switching circuit 104 is connected in parallel with the DC-DC converter 306. However, many of the previously proposed topologies, such as that in [19], are not cost effective because they utilize a large number of switches.

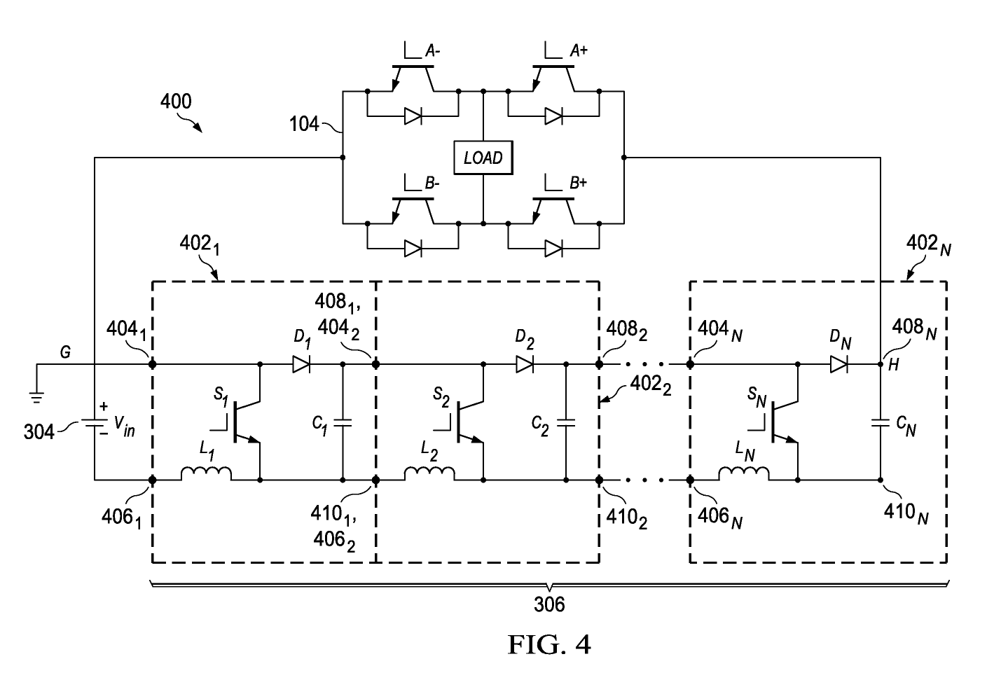

As it was previously mentioned, one suitable approach was introduced in [28] although the configuration in [28] does not allow bipolar pulse generation. Yet, with the addition of the H-bridge output stage the new topology of the present invention one can realize a broad set of pulse patterns, including the bipolar pulse needed to rupture algae cells. With this arrangement, the complete simplified schematic of the Marx generator 400 of the present invention is shown in FIG. 4. More specifically, the bipolar high-power pulse generator 400 includes a DC power source 304, a DC-DC converter 306 connected to the DC power source 304, and a H-bridge switching circuit 104 connected in parallel with the DC-DC converter 306. The H-bridge switching circuit 104 includes four switches (A+, A−, B+, B−) connected in a H configuration with a load 106 connected across the bridge. As shown, the H-bridge switching circuit 104 also includes a diode (D_{A+}, D_{A-}, D_{B+}, D_{B-}) connected in parallel with each switch (A+, A−, B+, B−). A controller (not shown) is connected to the DC-DC converter 306 and the H-bridge switches (A+, A−, B+, B−). The DC-DC converter 306 includes two or more boost cells (402₁, 402₂ . . . 402_{N}) connected together for multiplying the input voltage across the H-bridge terminal. Each boost cell 402ᵢ includes a positive input node 404ᵢ, a negative input node 406ᵢ, a switch Sᵢ connected in series with an inductor Lᵢ wherein the series connected switch Sᵢ and inductor Lᵢ are connected in parallel with the positive 404ᵢ and negative 404ᵢ input nodes, a diode Dᵢ connected in series with a capacitor Cᵢ wherein the series connected diode Dᵢ and capacitor Cᵢ are connected in parallel with the switch Sᵢ and the capacitor Cᵢ is connected in parallel with a positive output node 408ᵢ and a negative output node 410ᵢ. Since switches pulse durations are extremely short in comparison with the operating frequency, the input and output voltages of each boost stage are approximately equal and the inductors do not increase their currents in an appreciable way when the switches Sᵢ are closed. This characteristic is an important advantage because diodes reverse recovery losses are reduced significantly [28]. Therefore, the bipolar solid-state Marx generator 400 of the present invention as shown in FIG. 4 is capable of achieving the desired high-power flexible output, with higher efficiency, and lower cost than other proposed topologies.

An analysis of the bipolar solid-state Marx generator circuit 400 in accordance with one embodiment of the present invention will now be discussed. In order to analyze the steady state operations of the circuit indicated in FIG. 4, the following conditions are assumed during one switching cycle.

(i) The conduction intervals for the switches Sᵢ in each boost stage are short enough to ensure that the inductors are not significantly charged when these switches Sᵢ are on.

(ii) The switches Sᵢ in the cascaded boost high-voltage source stage are faster than the H-bridge switches (A+, A−, B+, B−).

(iii) The dead time t₃ is short.

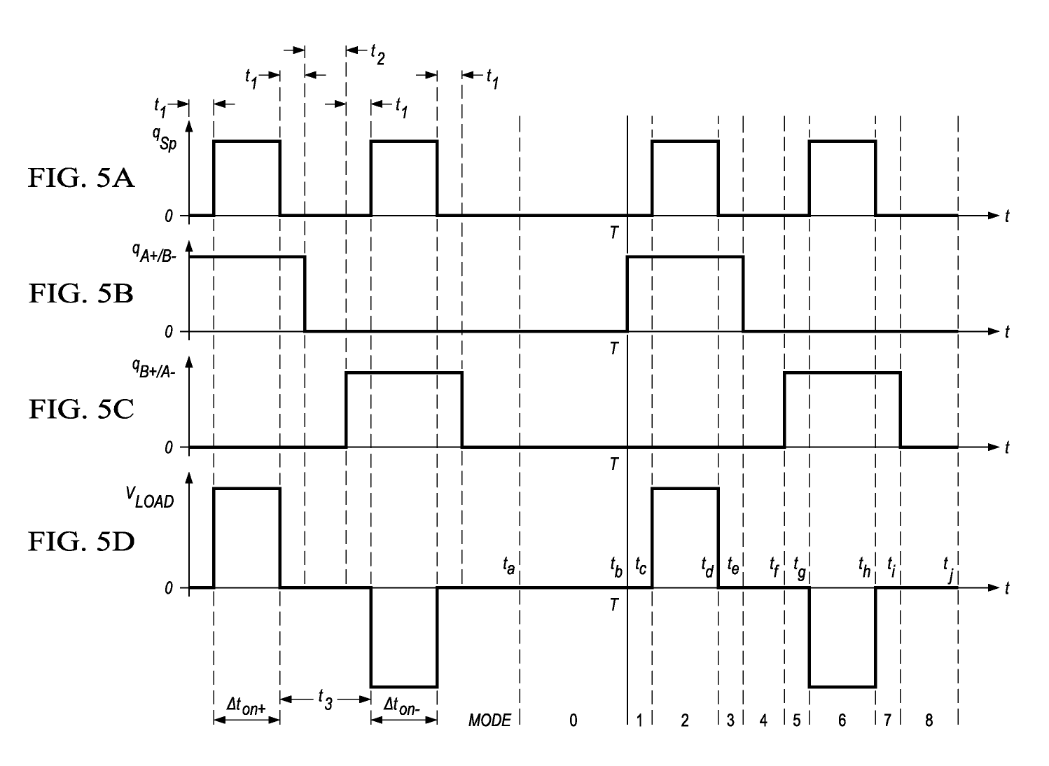

Based on these assumptions, the switching timing in FIGS. 2A-2D can be divided in nine different modes shown in FIGS. 5A-5D. As this figure indicates, the addition of the H-bridge output stage fundamentally alters the operational concept presented in [28] by requiring switching the switches Sᵢ twice in a switching period with a short interval in between these two pulses.

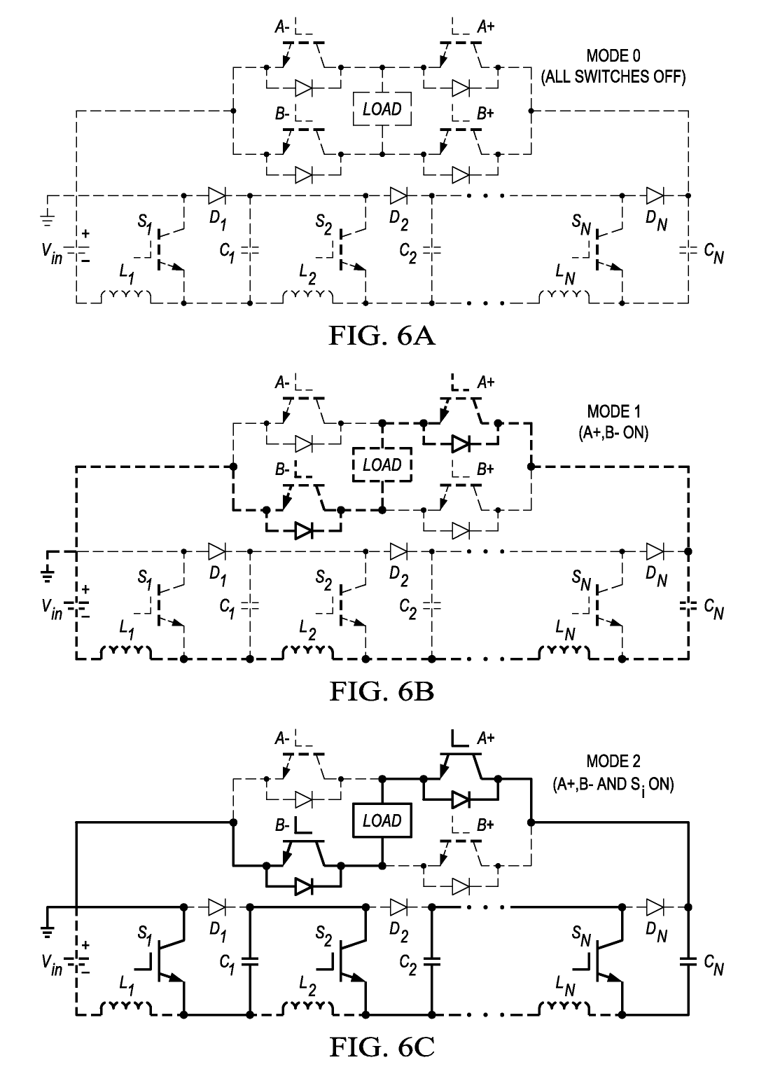

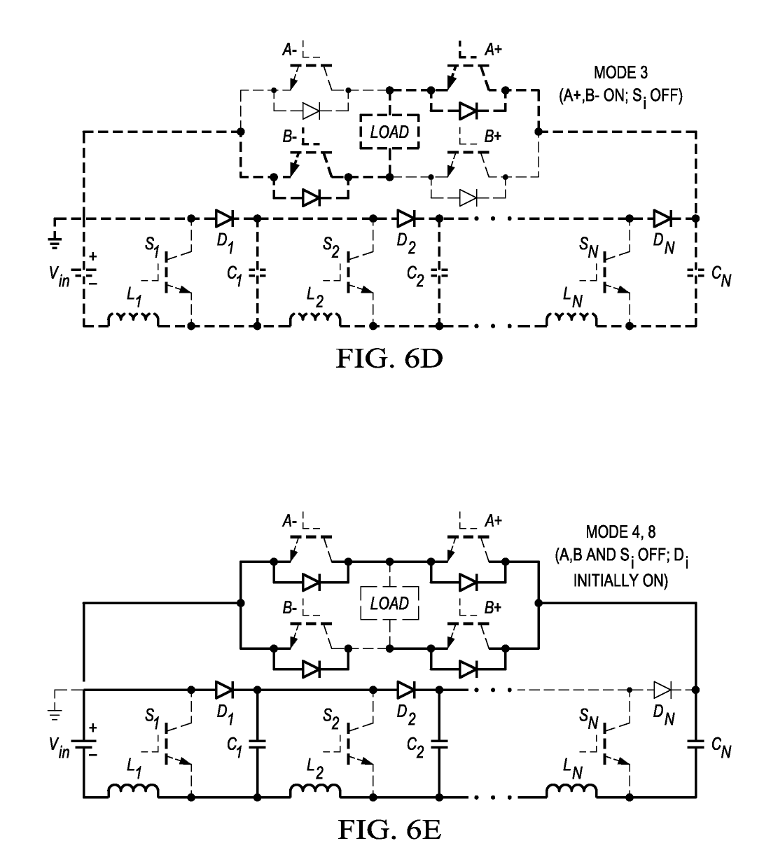

Now referring both to FIGS. 5A-5D and 6A-6E, the operation of the bipolar solid-state Marx generator circuit 400 can be described based on these nine operational modes:

Mode 0 (FIG. 6A; tₐ<t<t_{b})

It is assumed that initially all capacitors Cᵢ are charged to the input voltage. Hence, all the diodes Dᵢ are reverse biased and all the inductor Lᵢ currents are zero. In addition, all the semiconductor switches Sᵢ are commanded to be off. Since there is no current flow in the circuit, the output voltage is zero.

Mode 1 (FIG. 6B; t_{b}<t<t_{c})

For smooth operations in the H-bridge circuit, A+ and B− were turned on before connecting the capacitors Cᵢ in series. The equivalent circuit is indicated in FIG. 6B, in which the switches Sᵢ are off and the diodes Dᵢ are reverse biased. Since the capacitor C_{N} was maintained at the input voltage level there was still no current, and the output voltage was zero.

Mode 2 (FIG. 6C; t_{c}<t<t_{d})

As soon as the last of the Sᵢ switches turned on, a positive pulse was applied to the load. All the capacitors Cᵢ were connected in series because all the switches S₁˜S_{N}, A+, and B− were on at this time. The last switch among the switches Sᵢ to start conducting determined the starting edge of the positive pulse. Ideally, the pulsed output voltage vₒ would equal the sum of the N capacitor voltages. Since all N capacitors are charged up to the input voltage, then

vₒ=NVᵢₙ. (1)

Hence, for a primarily resistive load Rₒ as is the case with algae cells, the output current is

iₒ=NVᵢₙ/Rₒ. (2)

However, due to the presence of an equivalent capacitance

$${C}_{e} = \frac{1}{{\sum }_{j = 1}^{N} \frac{1}{{C}_{j}}} = \frac{{C}_{i}}{N} . \qquad ( 3 )$$

From the series connection of capacitors, the pulsed voltage is in reality subject to an exponential decay as indicated in

vₒ=NVᵢₙe^{-(t/C}^{ₑ}^{R}^{ₒ}⁾. (4)

In (3) Cᵢ represents any of the capacitances in the high voltage source circuit, which are all assumed to be equal. During this interval the inductor currents started to increase. However, due to their large inductances and short duration of this mode, the inductor Lᵢ currents increased only slightly. Although the inductor Lᵢ currents were very small, it can be expected that by the end of this interval corresponding to Mode 2 they would slightly differ among each other. This small difference led to voltage spikes when transitioning from Mode 2 to Mode 3 unless care was taken in selecting all inductors with high enough and as similar inductances as possible and in controlling the switching signals properly.

Mode 3 (FIG. 6D; t_{d}<t<tₑ)

For the same reasons explained previously, A+ and B− are switched off tᵢ seconds after all the Sᵢ switches are expected to be off. However, contrary to what happened in Mode 1, the output voltage although very low was not exactly zero because the primary DC voltage source 304 slightly charges all the inductors Lᵢ and the output capacitor C_{N} connected in an RLC series circuit with the load.

Mode 4 (FIG. 6E; tₑ<t<t_{f})

After the H-bridge switches A+ and B− were turned off, the diodes Dᵢ started to conduct, thus providing a path for the inductor Lᵢ currents. If the period of this stage was long enough, the capacitors Cᵢ would be charged to the input voltage level. However, based on assumption (iii) this time was short so the capacitors C are not charged and their voltages remain approximately equal to the voltage they had at t=t_{d}. For the same reason, although the inductor Lᵢ currents increased slightly, their initial and final values could be considered approximately equal. Since all switches (A+, A−, B+, B−) at the H-bridge were open, the output voltage is zero because there is no current at the load.

Mode 5, 6, and 7 (FIGS. 6B, 6C, and 6D)

Modes 5, 6, and 7 are the equivalent to Modes 1, 2, and 3, respectively, except that now A− and B+ switches are on instead of A+ and B−. Hence (1), (2), and (4) are replaced by

vₒ=−NVᵢₙ (5)

iₒ=−NVᵢₙ/Rₒ (6)

vₒ=−NVᵢₙe^{(-t/C}^{ₑ}^{R}^{ₒ}⁾ (7)

respectively.

Mode 8 (FIG. 6E; tₒ<t<t_{f})

State 8 was similar to state 4. However, now the duration of this mode was long enough to allow DC steady state conditions to settle in. Thus, from FIG. 6E, the capacitors Cᵢ were charged to the input voltage level and the inductors Lᵢ were completely discharged leading to the disappearance of all circuit currents. As in Mode 4, the output voltage equals zero.

Various design considerations related to the present invention that are well known to those skilled in the art will not be discussed. Inductors design is dependent on their current behavior during Mode 8 because this is the only mode when their currents could reach appreciable values. Although the analysis in [28] calculates an upper bound for the inductances by assuming that the boost stages operate in discontinuous conduction mode, the same approach can not be used here because each switch conducts twice during each switching period and those pulses are not evenly distributed in a switching period, and because the switching frequency is not high enough. Hence, linear approximations for inductor currents waveforms such as those used in [28] are not valid. In the proposed circuit suitable inductance values are selected so the input current during Mode 8 does not exceed the primary DC power source rating, and so all inductor currents become zero before the end of the switching period. Because of the many interactions among energy storage elements, it is difficult to obtain the inductor currents analytically from FIG. 6(e), so for the prototype discussed in the next section, the inductances values were chosen based on simulation results.

For the capacitances, Mode 2 is the determinant design mode because this is the interval when the capacitors Cᵢ are discharged. If an acceptable voltage drop is specified during the discharge, then the equivalent capacitance Cₑ yielded by the series connection of all capacitances in the circuit is

$${C}_{e} = \frac{{I}_{o} Δ {t}_{on}}{Δ v} \qquad ( 8 )$$

which assumes linear voltage changes—an assumption now valid because of Mode 2 short duration. In (8) Δtₒₙ equals Δtₒₙ₊ or Δt_{on -} shown in FIG. 5. Hence, from (3) and considering that Iₒ=Vₒ/Rₒ

$${C}_{i} = \frac{N Δ {t}_{on} {V}_{o}}{{R}_{o} Δ v} \qquad ( 9 )$$

and from (1)

$${C}_{i} = \frac{{N }^{2} Δ {t}_{on} {V}_{in}}{{R}_{o} Δ v} \qquad ( 10 )$$

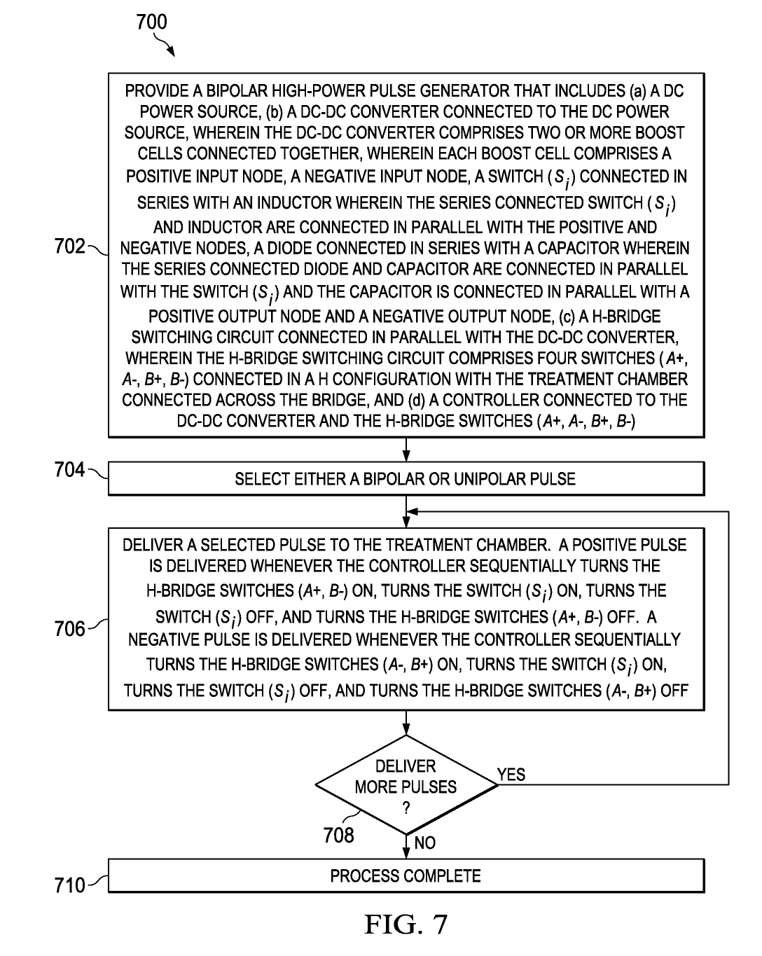

Now referring to FIG. 7, a method 700 of treating one or more biological cells, water, or a pumpable food within a treatment chamber is shown. A bipolar high-power pulse generator in provided in block 702. The bipolar high-power pulse generator includes (a) a DC power source, (b) a DC-DC converter connected to the DC power source, wherein the DC-DC converter comprises two or more boost cells connected together, wherein each boost cell comprises a positive input node, a negative input node, a switch (Sᵢ) connected in series with an inductor wherein the series connected switch (Sᵢ) and inductor are connected in parallel with the positive and negative nodes, a diode connected in series with a capacitor wherein the series connected diode and capacitor are connected in parallel with the switch (Sᵢ) and the capacitor is connected in parallel with a positive output node and a negative output node, (c) a H-bridge switching circuit connected in parallel with the DC-DC converter, wherein the H-bridge switching circuit comprises four switches (A+, A−, B+, B−) connected in a H configuration with the treatment chamber connected across the bridge, and (d) a controller connected to the DC-DC converter and the H-bridge switches (A+, A−, B+, B−). A type of pulse to be delivered to the treatment chamber is selected in block 704. The pulse characteristics selected may include voltage level, duration, frequency, number of pulses, pulse shape and pulse type (e.g., bipolar or unipolar). One or more pulses are delivered to the treatment chamber in block 706. A positive pulse is delivered whenever the controller sequentially turns the H-bridge switches (A+, B−) on, turns the switch (Sᵢ) on, turns the switch (Sᵢ) off, and turns the H-bridge switches (A+, B−) off, and/or (b) a negative pulse is delivered whenever the controller sequentially turns the H-bridge switches (A−, B+) on, turns the switch (Sᵢ) on, turns the switch (Sᵢ) off, and turns the H-bridge switches (A−, B+) off. If more pulses are to be delivered, as determined in decision block 708, the process returns to block 706 to deliver the selected pulses. If, however, no more pulses are to be delivered, as determined in decision block 708, the process is completed in block 710.

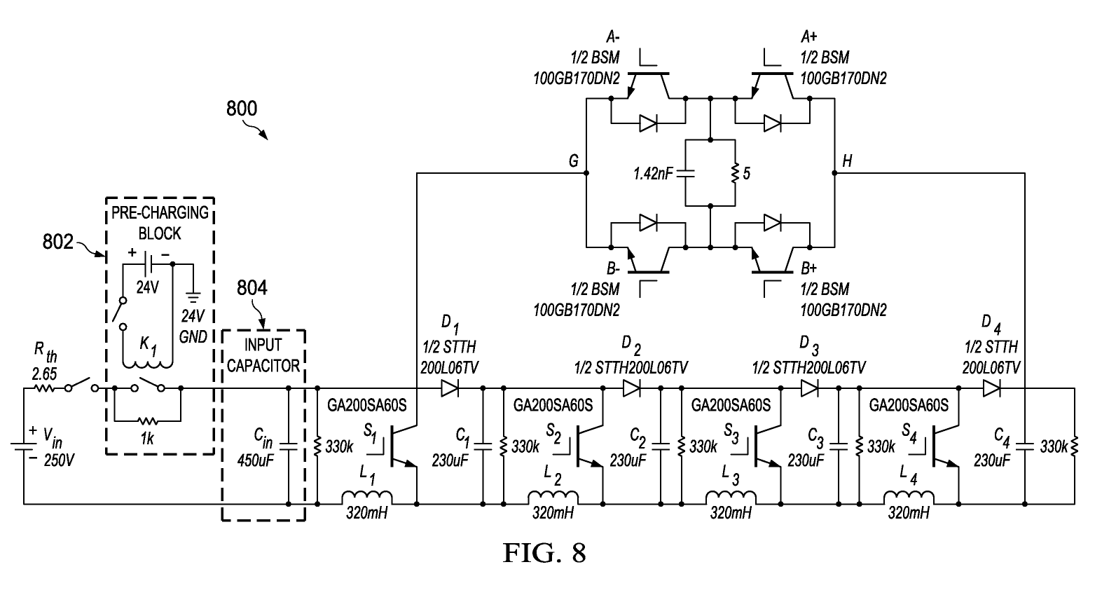

In order to verify the previous analysis, simulations and experimental tests were conducted with a 4-stages 1 kV/200 A prototype for the bipolar high-power pulse generator 800, such as the one represented in FIG. 8. For the simulations, the safety resistances Rᵢ in parallel with the capacitors the pre-charging circuit 802, and the small load capacitance 804 were omitted because of their negligible influence for circuit operation.

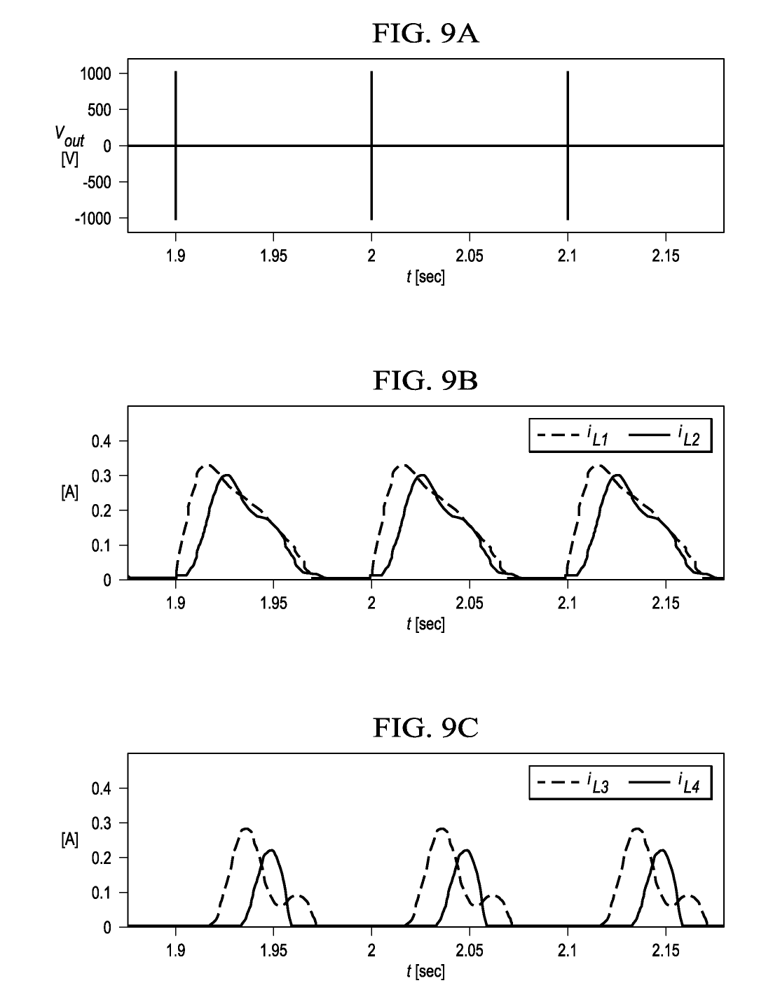

Simulations were conducted with a dual purpose: initially they were used to calculate adequate inductance values and then they were used to verify the analysis and circuit operation before building the prototype. The circuit was designed to be operated at 10 Hz, not because of limitation in the operating frequency, but because of needs involved with the algae oil extraction process. Similar limitations lead to the choice of a pulse-width Δtₒₙ of 8 μsec. with t₁=1 μsec. and t₂=3 μsec. As FIG. 8 indicates the load resistance equals 5Ω and Vᵢₙ equals 250 V leading to pulses with a 1 kV amplitude. The capacitances were selected to equal 230 μF resulting from (8) in a voltage drop of about 30 V. Since the maximum current allowed by the primary dc voltage source was 0.5 A the simulations indicated that inductors with L=320 mH were a suitable choice. FIGS. 9A-9C are graphs showing simulated output voltages and inductor currents for the bipolar solid state Marx generator shown in FIG. 7. As FIGS. 9B and 9C show, the input current stayed below 0.5 A and all inductor currents dropped to zero before the beginning of the next immediate switching period.

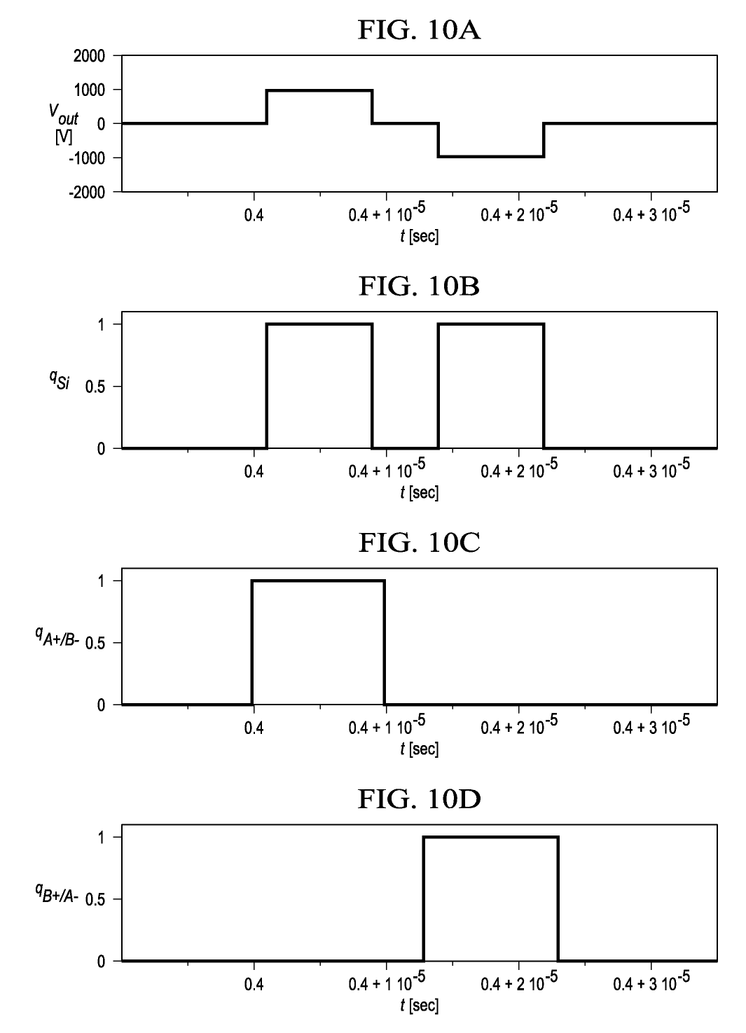

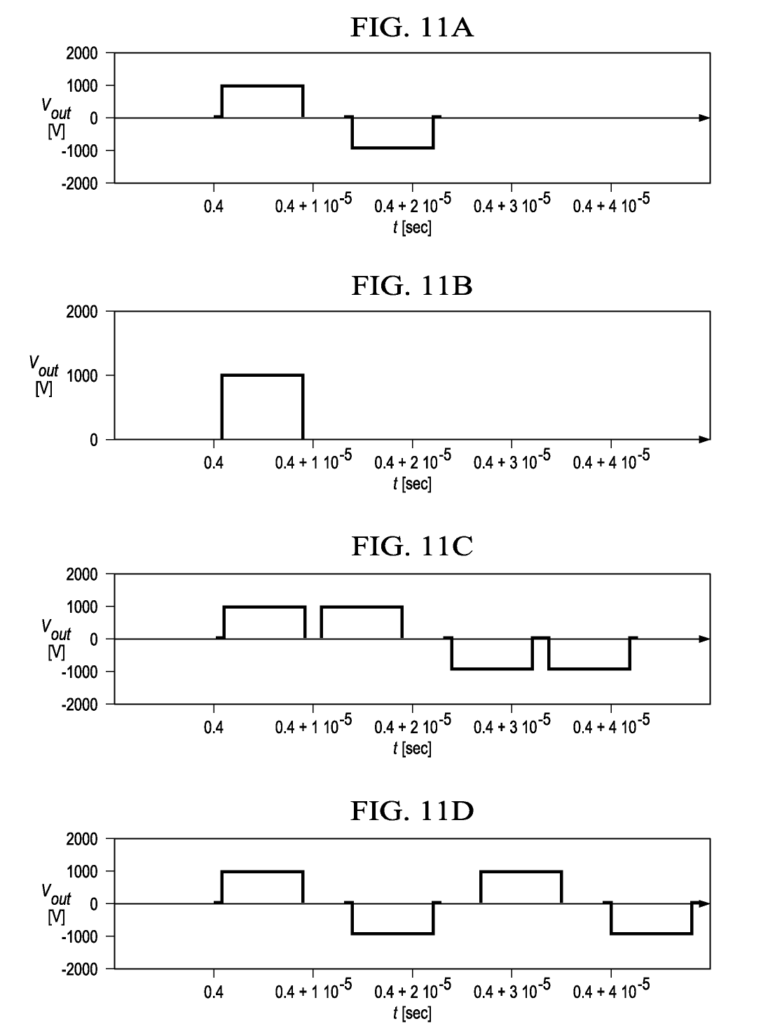

FIGS. 10A-10D show the simulation verification of the switching strategy. Simulations were used to test different switching patterns. As FIGS. 11A-11D exemplify, the Marx generator topology of the present invention is able to produce a variety of unipolar and bipolar pulses as well as train of pulses without any system reconfiguration.

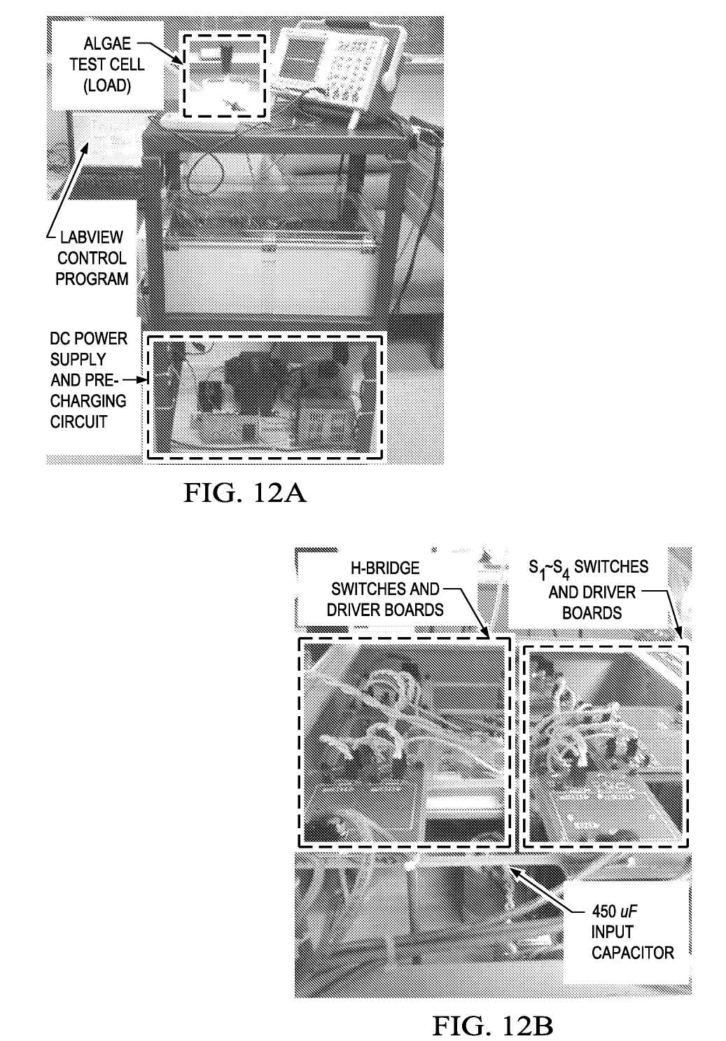

A prototype was built and tested. For the hardware prototype with schematic shown in FIG. 8 and experimental setup displayed in FIG. 12A with a close up of the circuit details in FIG. 12B, a 600V GA200SA60S IGBT was chosen for the switches Sᵢ and a 1.7 kV BSM100 GB170DN2 IGBT was selected to accommodate the 1 kV output voltage at the H-bridge. Polypropylene film capacitors were used for the 230 μF inductors. For safety, a 330 kΩ resistor was placed in parallel with each capacitor for self-discharge.

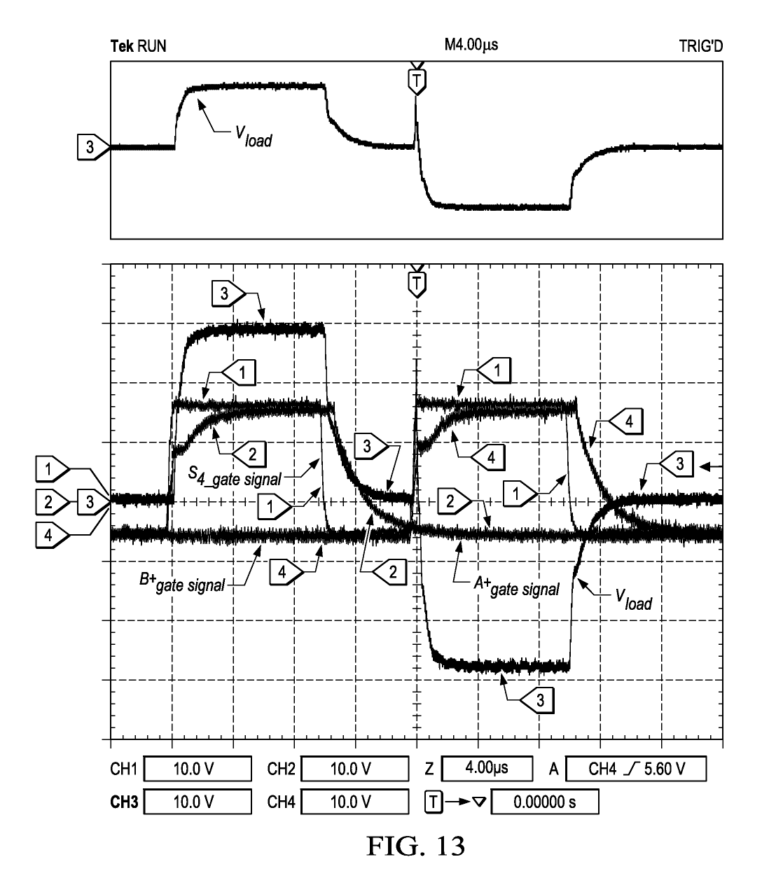

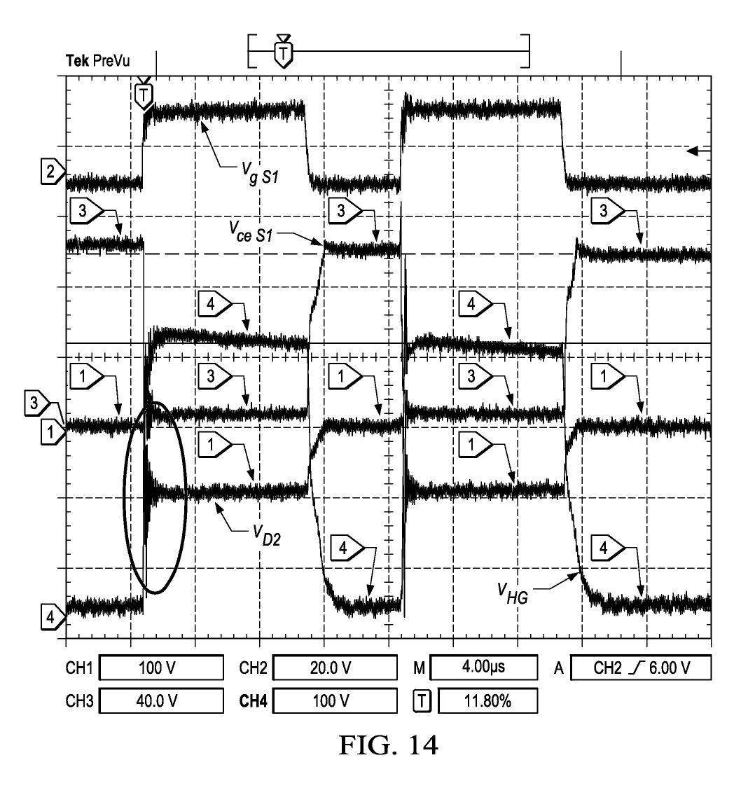

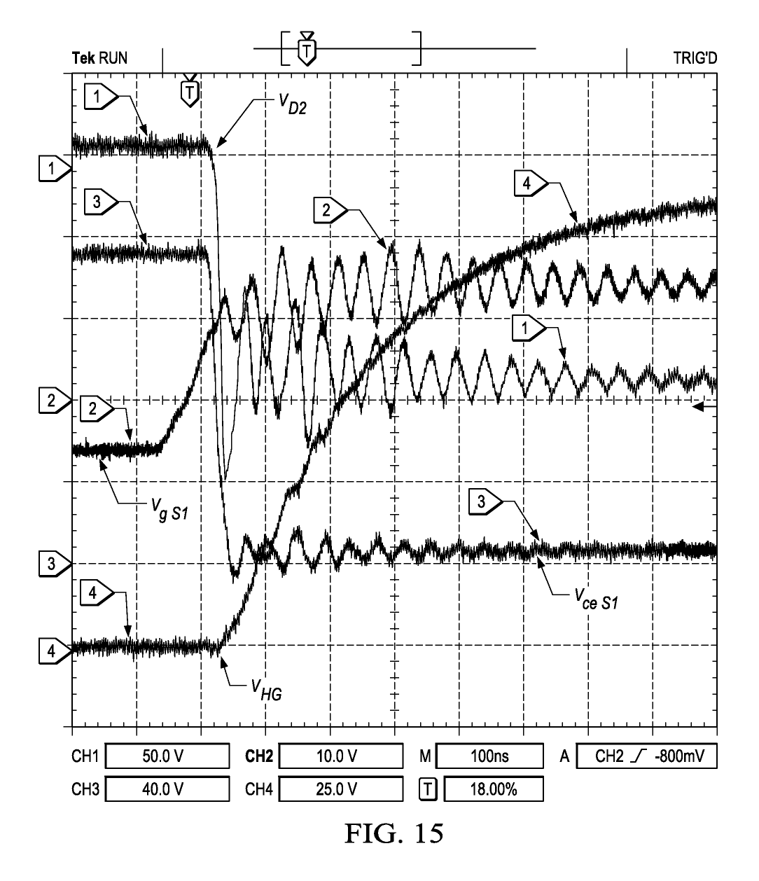

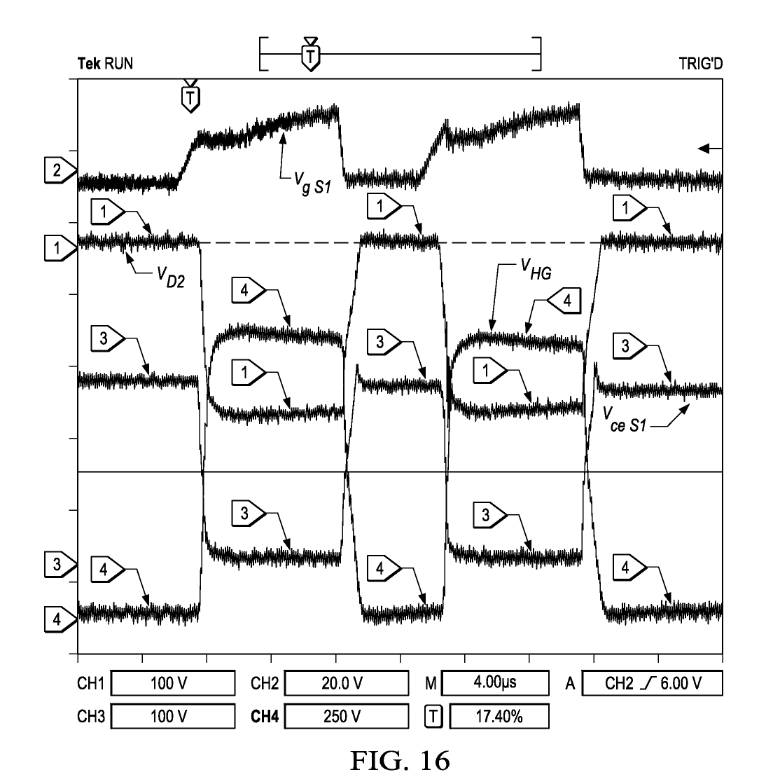

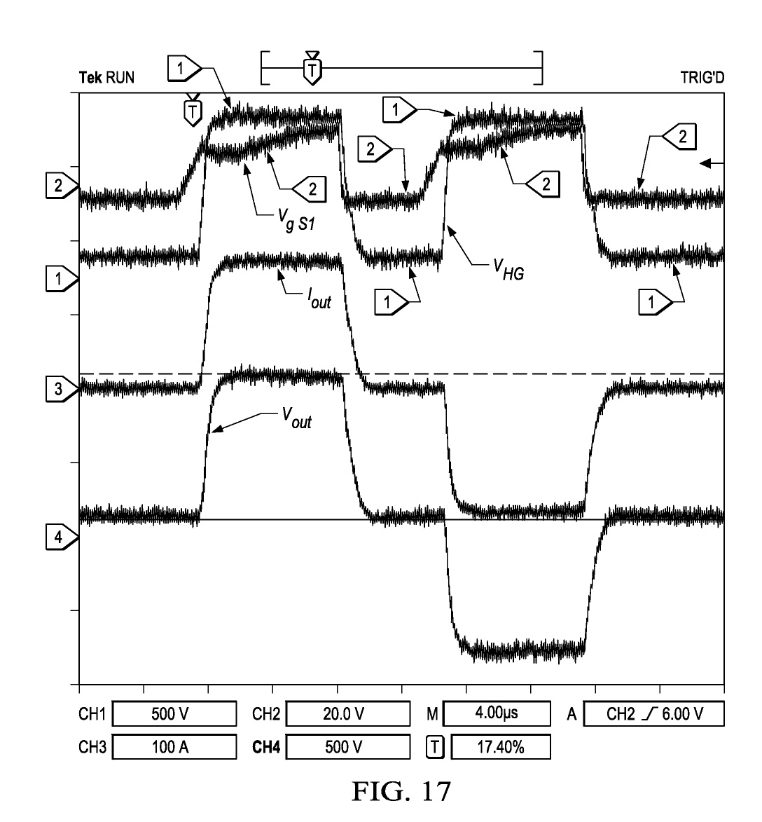

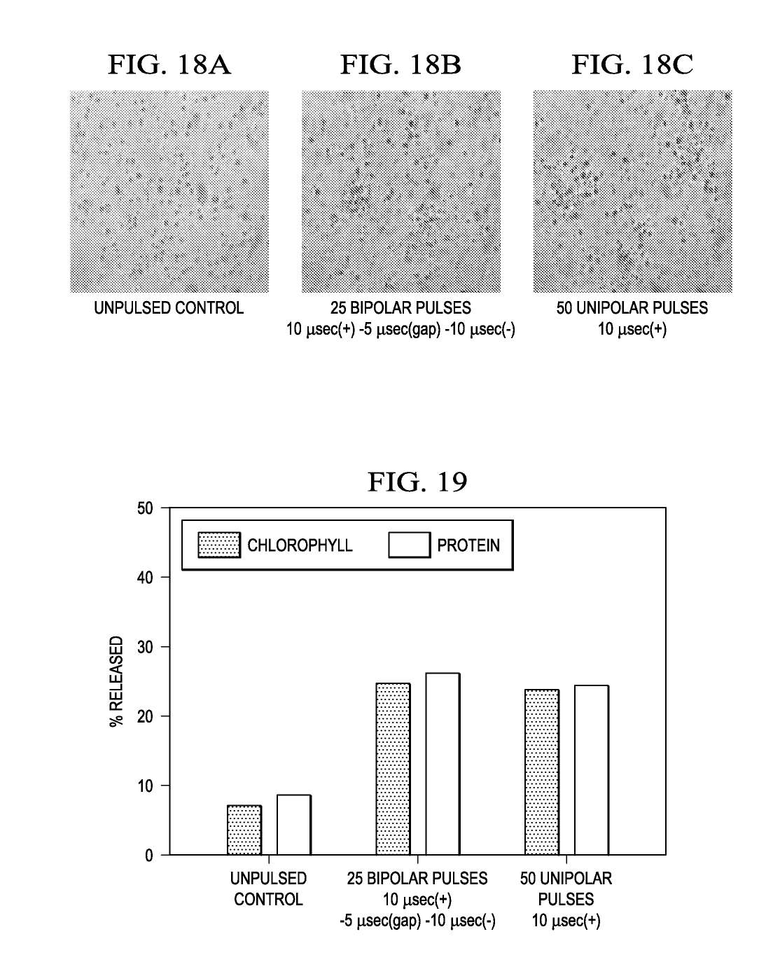

As described previously, the circuit of the present invention requires short time delays in between switching signals in order to avoid undesirable effects. An example of one of those effects is shown in FIG. 13. As FIG. 13 exemplifies, an insufficiently short choice for the delay time tᵢ does not prevent a positive voltage spike during the beginning of the negative pulse. In this case, dissimilar device properties makes the switches S₁˜S₄ to be turned off and almost simultaneously back on again before there was enough time for A+ and B− to fully stop conducting. Thus, a delay time t₁ was chosen according to the turn-on times of the IGBTs S₁˜S₄. Another practical issue leading to delays is shown in FIG. 14. As this figure indicates, initially there were over voltages on diodes and IGBTs caused by the interaction among circuit stray inductances and capacitances, the load's small capacitive component, and the fast rising edge of the gate driving signal (V_{g}Sᵢ) in the switches Sᵢ. Some ringing was also observed, particularly on the diode caused by its reverse recovery characteristics. These undesirable behaviors are shown with more detail in FIG. 15, which shows an expanded view of the red region surrounded by a red ellipse in FIG. 14. In FIGS. 14 to 17, V_{ce}S₁ represents the voltage across the collector and the emitter of S₁; VD₂ refers to the voltage across the diode D₂; and VHG means the voltage across the H-bridge shown in FIG. 4. These over voltages and ringing problems were solved when the gate driving signal of S₁ (V_{g}S₁) in FIG. 16 was slowed down with respect to that of S₁ (V_{g}S₁) in FIG. 8. As shown in FIG. 17, with this slower gate drive signal for the switches Sᵢ, it is possible to achieve the desired 1 kV/200 A bipolar pulsed output voltage. The pulses widths are about 10 μsec positive and 10 μsec negative whereas the dead time was about 5 μsec. Thus, FIG. 17 serves to experimentally verify the proposed circuit topology and analysis.

The prototype was then used on various biological samples. FIG. 18A-18C show the debris images for an unpulsed control sample (FIG. 18A), a sample after 25 bipolar pulses (FIG. 18B), a sample after 50 unipolar pulses (FIG. 18C). The upper three microscopic images in FIG. 18A-18C show increased debris in the bipolar pulsed (FIG. 18B) and the unipolar pulsed (FIG. 18C) samples compared to un-pulsed control sample (FIG. 18A). The bipolar case was configured as before with a 10 μsec positive pulse followed by a 5 μsec dead time and a 10 μsec negative thereafter. The unipolar pulse pattern is formed with a single positive 10 μsec pulse. The chart based on the images in FIG. 19 describes that the prototype pulse generator successfully ruptured algae membranes.

In the chart of FIG. 19, oil contents released from 25 bipolar pulses is a little more than the oil contents released from 50 unipolar pulses. Hence, as FIG. 19 indicates, for the same operating frequency the bipolar pulse pattern is able to rupture algae at double the rate than the unipolar pattern. For instance, at an operating frequency of 10 Hz 2.5 sec of operation under bipolar pulses ruptures a few more cells than 5 sec of operation under unipolar pulses. Even this result highlights an important advantage of bipolar pulse generators, particularly because there is a maximum operating frequency constraint by algae flow requirements, additional yield expected from the fast polarity reversal was not observed. This outcome is caused by the dead time between pulses with different polarities which may be long enough to allow cell membranes to recover their original state between pulses with opposing polarity. However, some works involving reversible processes, such as [8] and [10], seeming to suggest similar cell responses to bipolar pulses even without dead time, i.e. bipolar pulses produce better results than unipolar pulses but no additional cell destruction is observed from the output voltage fast polarity reversal. As can be seen from the foregoing discussion, the positive pulse width, the negative pulse width, the dead time between two pulses, and the operating frequency are adjustable and can be based on one or more circuit components, load characteristics or specifications.

This present disclosure describes and analyzes a bipolar pulse generator intended for algae cell oil production. Some additional potential applications include biology and plasma sciences, and food processing. The topology of the circuit of the present invention has fast rise time, rectangular pulse, and easy step-up input voltage. The circuit is also cost effective and avoids resistive losses found in conventional Marx generators. In addition, the circuit of the invention is extremely flexible, being able to produce different pulse patterns by control action and without having to reconnect any of the circuit elements or alter its topology. The steady-state analysis and design criteria of the bipolar pulse generator of the present invention are also described.

The analysis and circuit concept of the present invention were verified both with simulations and laboratory studies on a hardware prototype with a 1 kV/200 A bipolar solid-state pulsed generator. In addition, biological test results from processing algae with the fabricated prototype circuit verify that the circuit of the present invention is able to rupture cells and indicate that bipolar pulse patterns may yield twice the production rate than unipolar pulse configurations. Thus, from an electrical engineering perspective the circuit design achieves the desirable goals.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] J. C. Weaver and T. A. Chizmadzhev, “Theory of electroporation: A review,” Bioelectrochemistry and Bionergetics, vol. 41, issue 2, pp. 135-160, December 1996.
[2] M. Puc et. al., “Techniques of signal generation required for electropermeabilization. Survey of electropermeabilization devices.” Bioelectrochemistry, vol. 64, issue 2, pp. 113-124, September 2004.
[3] T. Y. Song, “Electroporation of cell membranes.” Biophysical Journal; 60(2): pp. 297-306, August 1991.
[4] D. C. Chang, “Cell poration and cell fusion using an oscillating electric field.” Biophysical Journal, vol. 56, issue 4, pp. 641-652, October 1989.
[5] U. Zimmerman, G. Pilwat, and F. Riemann, “Dielectric breakdown of cell membranes,” Biophysical Journal, vol. 14, pp. 881-899, 1974.
[6] U. Zimmerman, F. Beckers, and H. G. L. Coster, “The effect of pressure on the electrical breakdown in the membranes of Valonia Utricularis,” Biochimica et. Biophysica Acta, vol. 464, pp. 399-416, 1977.
[7] A. Angersbach, V. Heinz, and D. Knorr, “Effects of pulsed electric fields on cell membranes in real food systems,” Innovative Food Science and Emerging Technologies, vol. 1, no. 2, pp. 135-149(15), June 2000.
[8] E. Tekle, R. Dean Astumiant and P. Boon Chock, “Electroporation by using bipolar oscillating electric field: An improved method for DNA transfection of NIH 3T3 cells.” Proc. National Academy of Science, vol. 88, pp. 4230-4234, May 1991.
[9] Q. Bai-Lin, Z. Qinghua, G. V. Barbosa-Canovas, B. G. Swanson, and P. D. Pedrow, “Inactivation of microorganisms by pulsed electric fields of different voltage waveforms,” Dielectrics and Electrical Insulation, IEEE Transactions on, vol. 1, pp. 1047-1057, 1994.
[10] T. Kotnik, et. al., “Cell membrane electropermeabilization by symmetrical bipolar rectangular pulses: Part I. Increased efficiency of permeabilization.” Bioelectrochemistry, vol. 54, issue 1, pp. 83-90, August 2001.
[11] S. Töpfl, “Pulsed Electric Fields (PEF) for Permeabilization of Cell Membranes in Food- and Bioprocessing—Applications, Process and Equipment Design and Cost Analysis.” Ph.D. Dissertation, Berlin University of Technology, Berlin, Germany, September 2006.
[12]E. Marx, Verfahren zur Schlagprüfung von Isolatoren and anderen elektrischen Vorrichtungen, German Patent #455933, 1923.
[13] T. Heeren, et. al., “Novel Dual Marx Generator for Microplasma Applications,” EEE Transactions on Plasma Science, vol. 33, no. 4, pp. 1205-1209, August 2005.
[14] S. Singer, “Transformer description of a family of switched systems.” IEE Proceedings of Electronic Circuits and Systems, vol. 129, issue 5, pp. 205-210, October 1982.
[15]H. Li, et. al, “Development of Rectangle-Pulse Marx Generator Based on PFN.” in IEEE Transactions on Plasma Science, vol. 37, no. 1, pp. 190-194, January 2009.
[16] K. Jong-Hyun, L. Sang-Cheol, L. Byoung-Kuk, S. V. Shenderey, K. Jong-Soo, and R. Geun-Hie, “A high-voltage bi-polar pulse generator a using push-pull inverter,” in Industrial Electronics Society, 2003., vol. 1, pp. 102-107.
[17] J. H. Kim, I. W. Jeong, H. J. Ryoo, S. Shenderey, J. S. Kim, and G. H. Rim, “Semiconductor switch-based fast high-voltage pulse generators,” in Pulsed Power Conference, 2003. Digest of Technical Papers. PPC-2003. 14th IEEE International, 2003, pp. 665-668 Vol. 1.
[18]H. Canacsinh, L. M. Redondo, and J. F. Silva, “New solid-state Marx topology for bipolar repetitive high-voltage pulses,” in Power Electronics Specialists Conference, 2008. PESC 2008. IEEE, 2008, pp. 791-795.
[19] L. M. Redondo, H. Canacsinh, and J. F. Silva, “Generalized Solid-state Marx Modulator Topology,” IEEE Transactions on Dielectrics and Electrical Insulation, vol. 16, pp. 1037-1042, August 2009.
[20] J. H. Kim, M. H. Ryu, B. D. Min, S. S. V., J. S. Kim, and G. H. Rim, “High voltage pulse power supply using Marx generator & solid-state switches,” in Industrial Electronics Society, 2005. pp. 1244-1247.
[21] L. M. Redono, J. F. Silva, P. Tavares, and E. Margato, “All Silicon Marx-bank Topology for High-voltage, High-frequency Rectangular Pulses,” in Power Electronics Specialists Conference, 2005. PESC '05. IEEE 36th, 2005, pp. 1170-1174.
[22] L. M. Redondo and J. F. Silva, “Repetitive High-Voltage Solid-State Marx Modulator Design for Various Load Conditions,” IEEE Transactions on Plasma Science, vol. 37, no. 8, pp. 1632-1637, August 2009
[23] Y. Wu, et. al., “Repetitive and High Voltage Marx Generator Using Solid-state Devices.” IEEE Transactions on Dielectrics and Electrical Insulation, vol. 14, no. 4, pp. 937-940, August 2007
[24] L. M. Redondo et. al., “Solid-state Marx Generator Design with an Energy Recovery Reset Circuit for Output Transformer Association,” in Rec. PESC 2007, pp. 2987-2991.
[25] J. H. Kim, J. S. Kim, S. Shenderey, and G. H. Rim, “High Voltage Marx Generator Implementation using IGBT Stacks.” IEEE Transactions on Dielectrics and Electrical Insulation, vol. 14, No. 4; pp. 931-936, August 2007
[26] W. D. Keith, L. J. Harris, L. Hudson, and M. W. Griffiths, “Pulsed electric fields as a processing alternative for microbial reduction in spice,” Food Research International, vol. 30, pp. 185-191, May 1997.
[27] M. Petkovsek, P. Zajec, J. Nastran, and D. Voncina, “Multilevel bipolar high voltage pulse source—interlock dead time reduction,” in EUROCON 2003. vol. 2, pp. 240-243.
[28] J. W. Baek, D. W. Yoo, G. H. Rim, and J. S. Lai, “Solid State Marx Generator Using Series-Connected IGBTs,” IEEE Transactions on Plasma Science, vol. 33, pp. 1198-1204, August 2005.

What is claimed is:

1. A bipolar high-power pulse generator comprising:
a DC power source;
a DC-DC converter connected to the DC power source;
a H-bridge switching circuit connected in parallel with the DC-DC converter, wherein the H-bridge switching circuit comprises four switches (A+, A−, B+, B−) connected in a H configuration with a load connected across the bridge; and
a controller connected to the DC-DC converter and the H-bridge switches (A+, A−, B+, B−).

2. The generator as recited in claim 1, wherein the DC-DC converter comprises two or more boost cells connected together, wherein each boost cell comprises a positive input node, a negative input node, a switch (Sᵢ) connected in series with an inductor wherein the series connected switch (Sᵢ) and inductor are connected in parallel with the positive and negative nodes, a diode connected in series with a capacitor wherein the series connected diode and capacitor are connected in parallel with the switch (Sᵢ) and the capacitor is connected in parallel with a positive output node and a negative output node.

3. The generator as recited in claim 2, wherein each capacitance of the capacitors in the boost cells comprises
$${C}_{i} = \frac{{N }^{2} Δ {t}_{on} {V}_{in}}{{R}_{o} Δ v} .$$

4. The generator as recited in claim 2, wherein:
a positive pulse is delivered to the load whenever the switch (Sᵢ) is on, the H-bridge switches (A+, B−) are on, and the H-bridge switches (A−, B+) are off; and
a negative pulse is delivered to the load whenever the switch (Sᵢ) is on, the H-bridge switches (A−, B+) are on, and the H-bridge switches (A+, B−) are off

5. The generator as recited in claim 4, wherein a positive pulse width, a negative pulse width, a dead time between two pulses, and an operating frequency are adjustable.

6. The generator as recited in claim 2, wherein the generator is operated in a series of stages comprising:
a stage zero comprising the switch (Sᵢ) is off and the H-bridge switches (A+, A−, B+, B−) are off;
a stage one comprising the switch (Sᵢ) is off, the H-bridge switches (A+, B−) are on, and the H-bridge switches (A−, B+) are off;
a stage two comprising the switch (Sᵢ) is on, the H-bridge switches (A+, B−) are on, the H-bridge switches (A−, B+) are off, and a positive pulse is delivered to the load;
a stage three comprising the switch (Sᵢ) is off, the H-bridge switches (A+, B−) are on, and the H-bridge switches (A−, B+) are off;
a stage four comprising the switch (Sᵢ) is off, the H-bridge switches (A+, A−, B+, B−) are off, and the diode is initially on;
a stage five comprising the switch (Sᵢ) is off, the H-bridge switches (A−, B+) are on, and the H-bridge switches (A+, B−) are off;
a stage six comprising the switch (Sᵢ) is on, the H-bridge switches (A−, B+) are on, the H-bridge switches (A+, B−) are off, and a negative pulse is delivered to the load;
a stage seven comprising the switch (Sᵢ) is off, the H-bridge switches (A−, B+) are on, and the H-bridge switches (A+, B−) are off; and
a stage eight comprising the switch (Sᵢ) is off, the H-bridge switches (A+, A−, B+, B−) are off, and the diode is initially on.

7. The generator as recited in claim 1, further comprising a diode (D_{A+}, D_{A-}, D_{B+}, D_{B-}) connected in parallel with each switch (A+, A−, B+, B−) in the H-bridge switching circuit.

8. The generator as recited in claim 1, wherein the DC power supply comprises:
a DC voltage source;
a power supply resistor connected in series with the DC voltage source; and
a power supply switch connected in series with the resistor.

9. The generator as recited in claim 1, further comprising a pre-charging circuit connected in series between the DC power source and the DC-DC converter;

10. The generator as recited in claim 1, further comprising an input capacitor connected in parallel with the DC power source between the DC power source and the DC-DC converter.

11. The generator as recited in claim 1, wherein the load comprises a pulse electric field (PEF) treatment chamber.

12. The generator as recited in claim 11, wherein the PEF treatment chamber is part of a constant flow treatment process.

13. The generator as recited in claim 11, wherein the PEF treatment chamber contains one or more biological cells, water, or a pumpable food.

14. The generator as recited in claim 12, wherein the one or more biological cells comprise bacterial cells, viral cells, algal cells, protozoal cells, plant cells, mammalian cells, animal cells or any combinations thereof.

15. The generator as recited in claim 14, wherein the algal cells on lysis release oil.

16. The generator as recited in claim 1, wherein the controller comprises a signal generator or a computer.

17. The generator as recited in claim 1, wherein the controller operates the generator in a unipolar pulse mode or a bipolar pulse mode.

18. A method of treating one or more biological cells, water, or a pumpable food within a treatment chamber comprising the steps of:
providing a bipolar high-power pulse generator comprising (a) a DC power source, (b) a DC-DC converter connected to the DC power source, wherein the DC-DC converter comprises two or more boost cells connected together, wherein each boost cell comprises a positive input node, a negative input node, a switch (Sᵢ) connected in series with an inductor wherein the series connected switch (Sᵢ) and inductor are connected in parallel with the positive and negative nodes, a diode connected in series with a capacitor wherein the series connected diode and capacitor are connected in parallel with the switch (Sᵢ) and the capacitor is connected in parallel with a positive output node and a negative output node, (c) a H-bridge switching circuit connected in parallel with the DC-DC converter, wherein the H-bridge switching circuit comprises four switches (A+, A−, B+, B−) connected in a H configuration with the treatment chamber connected across the bridge, and (d) a controller connected to the DC-DC converter and the H-bridge switches (A+, A−, B+, B−); and
delivering one or more pulses to the treatment chamber, wherein (a) a positive pulse is delivered whenever the controller sequentially turns the H-bridge switches (A+, B−) on, turns the switch (Sᵢ) on, turns the switch (Sᵢ) off, and turns the H-bridge switches (A+, B−) off, and/or (b) a negative pulse is delivered whenever the controller sequentially turns the H-bridge switches (A−, B+) on, turns the switch (Sᵢ) on, turns the switch (Sᵢ) off, and turns the H-bridge switches (A−, B+) off.

19. The method as recited in claim 18, wherein each capacitance of the capacitors in the boost cells comprises
$${C}_{i} = \frac{{N }^{2} Δ {t}_{on} {V}_{in}}{{R}_{o} Δ v} .$$

20. The method as recited in claim 18, wherein a positive pulse width, a negative pulse width, a dead time between two pulses, and an operating frequency are adjustable.

21. The method as recited in claim 18, further comprising a diode (D_{A+}, D_{A-}, D_{B+}, D_{B-}) connected in parallel with each switch (A+, A−, B+, B−) in the H-bridge switching circuit.

22. The method as recited in claim 18, wherein the DC power supply comprises:
a DC voltage source;
a power supply resistor connected in series with the DC voltage source; and
a power supply switch connected in series with the resistor.

23. The method as recited in claim 18, further comprising a pre-charging circuit connected in series between the DC power source and the DC-DC converter;

24. The method as recited in claim 18, further comprising an input capacitor connected in parallel with the DC power source between the DC power source and the DC-DC converter.

25. The method as recited in claim 18, wherein the treatment chamber is part of a constant flow treatment process.

26. The method as recited in claim 18, wherein the one or more biological cells comprise bacterial cells, viral cells, algal cells, protozoal cells, plant cells, mammalian cells, animal cells or any combinations thereof.

27. The method as recited in claim 26, wherein the algal cells on lysis release oil.

28. The method as recited in claim 18, wherein the controller comprises a signal generator or a computer.
